(19) United States
(12) Patent Application Publication
(10) Pub. No.: US 2024/0404670 A1
(43) Pub. Date: Dec. 5, 2024

(54) MEDICAL INFORMATION PROCESSING SYSTEM, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Katsuhiko FUJIMOTO, Saitama (JP); Satoshi IKEDA, Yaita (JP)

(73) Assignee: CANON MEDICAL SYSTEMS CORPORATION, Otawara-shi (JP)

(21) Appl. No.: 18/678,102

(22) Filed: May 30, 2024

(30) Foreign Application Priority Data
May 30, 2023 (JP) 2023-088739

(51) Int. Cl. G16H 20/00; G06T 17/00; G16H 50/20

(52) U.S. Cl. CPC G16H 20/00; G06T 17/00; G16H 50/20

(57) ABSTRACT

A medical information processing apparatus includes processing circuitry configured to acquire virtual medical consultation information generated by virtual medical consultation for a user from a digital twin hospital in a virtual space, wherein the virtual medical consultation is performed based on information regarding a real hospital corresponding to the digital twin hospital and medical-related information of the user, generate analysis information based on a plurality of pieces of the virtual medical consultation information acquired from a plurality of the digital twin hospitals, and present the analysis information to the user.

### CROSS REFERENCE TO RELATED APPLICATION

This application is based upon and claims the benefit of priority from the prior Japanese Patent Application No. 2023-088739, filed on May 30, 2023, the entire contents of which are incorporated herein by reference.

### FIELD

Embodiments disclosed in the present specification and drawings relate to a medical information processing system, a medical information processing method, and a storage medium.

### BACKGROUND

Various businesses using a virtual space such as a metaverse have been studied. In medical fields, it is considered to experience a hospital tour in a virtual space before actually visiting, and to provide a simulated experience of community and treatment for patients who have difficulty in going out.

In addition, there is known a health management system in which a user can ask questions and receive guidance from a doctor's avatar in a hospital set up in a virtual space.

### BRIEF DESCRIPTION OF THE DRAWINGS

FIG. 1 is a view illustrating an example of a configuration of a medical information processing system according to an embodiment;

FIG. 2 is a block diagram illustrating an example of a configuration of a server according to the embodiment;

FIG. 3 is a block diagram illustrating an example of a configuration of a terminal according to the embodiment;

FIG. 4 is a flowchart illustrating an example of a processing content executed by the medical information processing system according to the embodiment;

FIG. 5A is a view illustrating a first example of an output screen displayed on the terminal according to the embodiment;

FIG. 5B is a view illustrating a second example of the output screen displayed on the terminal according to the embodiment;

FIG. 5C is a view illustrating a third example of the output screen displayed on the terminal according to the embodiment; and

FIG. 5D is a view illustrating a fourth example of the output screen displayed on the terminal according to the embodiment.

### DETAILED DESCRIPTION

Hereinafter, a medical information processing system according to an embodiment will be described with reference to the drawings. Note that, in the following description, constituent elements having substantially the same functions and configurations are denoted by the same reference numerals, and redundant description will be made only when necessary.

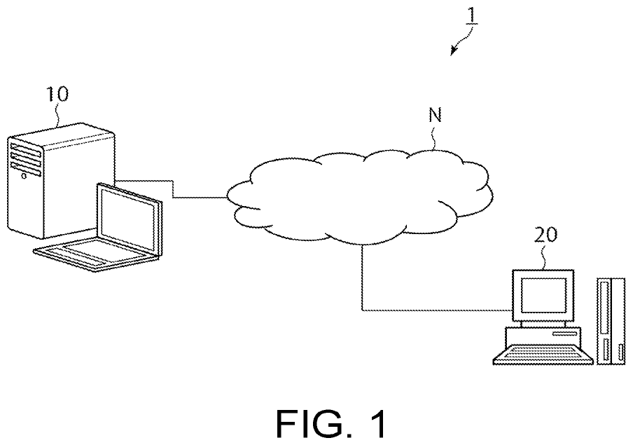

FIG. 1 is a view illustrating an example of a configuration of a medical information processing system 1 according to an embodiment. As illustrated in FIG. 1, the medical information processing system 1 according to the present embodiment includes a server 10 and a terminal 20 used by a user (patients, consultants, and the like). The server 10 and the terminal 20 are communicably connected to each other via a communication network N. Note that, a plurality of the terminals 20 may be connected to the server 10. Furthermore, the server 10 may be connected to another system such as an electronic medical record system that stores data such as a personal health record (PHR) of a user.

As will be described in detail below, the medical information processing system 1 according to the present embodiment is configured to provide medical-related information of a user (patient or the like) to a digital twin hospital (DTH) set up in a virtual space such as a metaverse space, perform virtual medical consultation, compare and analyze virtual medical consultation information including a result of virtual medical consultation obtained from the digital twin hospital, and provide an analysis result to the user. The digital twin hospital is a digital twin of a real hospital, and has digital data reflecting medical resources such as doctors, medical staff, and medical facilities of a corresponding real hospital, a medical technology (treatment handling ability), and the like. Note that, the user can take a virtual medical consultation by operating an anonymous avatar created in the virtual space.

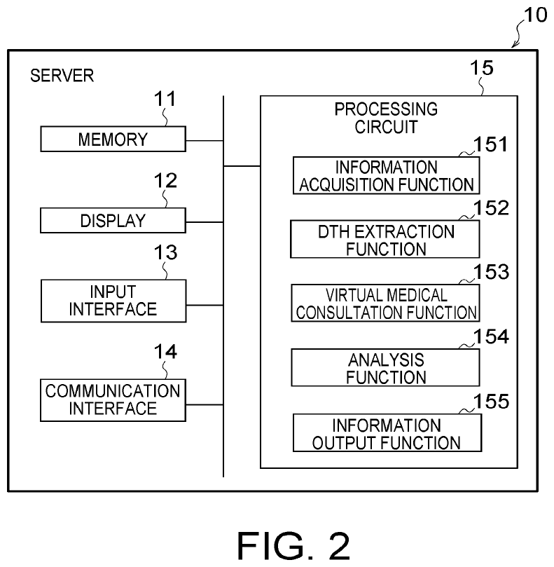

As illustrated in FIG. 2, the server 10 includes a memory 11, a display 12, an input interface 13, a communication interface 14, and a processing circuit 15. Hereinafter, details of each configuration of the server 10 will be described.

The memory 11 is connected to the processing circuit 15 and stores various types of information used in the processing circuit 15. The memory 11 is realized by, for example, a random access memory (RAM), a semiconductor memory element such as a flash memory, a hard disk, an optical disc, or the like.

The memory 11 stores various programs necessary for the processing circuit 15 to execute each function, various pieces of data processed by the programs, and the like. Note that, the various types of data handled in the present specification are typically digital data. Note that, medical-related information (described later) for each user may be stored in the memory 11.

The display 12 displays various images and information. For example, the display 12 displays a graphical user interface (GUI) or the like for receiving various operations. In the present embodiment, the display 12 is configured by, for example, a liquid crystal display, a cathode ray tube (CRT) display, or the like.

The input interface 13 receives various input operations, converts the received input operations into electrical signals, and outputs the electrical signals to the processing circuit 15. The input interface 13 is realized by, for example, a mouse, a keyboard, a touch panel, a trackball, a manual switch, a foot switch, a button, a joystick, or the like.

The communication interface 14 realizes communication with other devices via an external communication network such as the Internet according to various communication protocols. The type of the communication protocol to which the communication interface 14 conforms is not particularly limited. In the present embodiment, the server 10 communicates with the terminal 20 via the communication interface 14. Furthermore, the server 10 may communicate with the real hospital corresponding to the digital twin hospital via the communication interface 14.

The processing circuit 15 is an arithmetic circuit that performs various calculations and controls the operation of the server 10. The processing circuit 15 extracts a plurality of digital twin hospitals, for example, based on a DTH selection condition set by the user, and discloses the medical-related information of the user to the plurality of extracted digital twin hospitals. Then, after receiving the result of the virtual visit from each digital twin hospital, the received result is compared and analyzed to generate analysis information, and the analysis information is output to the terminal 20.

In order to realize such a function, the processing circuit 15 has an information acquisition function 151, a DTH extraction function 152, a virtual medical consultation function 153, an analysis function 154, and an information output function 155 as illustrated in FIG. 2. The information acquisition function 151 is an example of an information acquisition unit, the DTH extraction function 152 is an example of a DTH extraction unit, the virtual medical consultation function 153 is an example of a virtual medical consultation unit, the analysis function 154 is an example of an analysis unit, and the information output function 155 is an example of an information output unit.

In the present embodiment, each processing function executed by the information acquisition function 151, the DTH extraction function 152, the virtual medical consultation function 153, the analysis function 154, and the information output function 155 is stored in the memory 11 in a form of a program executable by a computer. The processing circuit 15 is a processor, and implements a function corresponding to each program by reading and executing the program from the memory 11. In other words, the processing circuit 15 in a state of reading each program has each function illustrated in the processing circuit 15 of FIG. 2.

Note that, FIG. 2 illustrates a case where each processing function of the information acquisition function 151, the DTH extraction function 152, the virtual medical consultation function 153, the analysis function 154, and the information output function 155 is implemented by a single processing circuit 15, but the embodiment is not limited thereto. For example, the processing circuit 15 may be configured by combining a plurality of independent processors, and each processor may implement each processing function by executing each program. Each processing function of the processing circuit 15 may be realized by being appropriately distributed or integrated into a single or a plurality of processing circuits.

Next, details of each function of the processing circuit 15 will be described.

The information acquisition function 151 acquires various types of information from the memory 11, the terminal 20, an external server system, and the like. For example, the information acquisition function 151 acquires information (a DTH selection condition or the like) input by the user from the terminal 20. Furthermore, the information acquisition function 151 acquires the medical-related information of the user from the terminal 20. Note that, the medical-related information may be acquired from an external server system.

The DTH selection condition is a condition set by the user to extract at least one digital twin hospital from a plurality of digital twin hospitals in the virtual space. The DTH selection condition is, for example, a location where a real hospital corresponding to the digital twin hospital exists (prefecture name, town name, and the like), a hospital visit time, facility conditions, cost, and the like. Note that, the DTH selection condition may be a condition related to hospital's reputation (including reviews of other people having similar symptoms), treatment cost, and the like.

The medical-related information is information including at least one of a user's chief complaint, medical information, biological information, and behavior information. The medical information is information including information regarding a medical examination result of the user, a past medical consultation result in a real hospital (a diagnosis result, an inspection result, etc.), a medication history, and the like. The biological information is information such as a body temperature, a blood pressure, and a heart rate, and is, for example, information acquired by a wearable sensor. The behavior information is information regarding a behavior such as a movement history (for example, a moving distance and the number of steps per day) of the user.

The DTH extraction function 152 extracts at least one digital twin hospital that performs virtual medical consultation for the user among a plurality of digital twin hospitals set up in the virtual space based on the DTH selection condition and/or the medical-related information. For example, the DTH extraction function 152 extracts a digital twin hospital having a real hospital in an area designated by the user and having a clinical department corresponding to a user's main complaint. Note that, in a case where the user designates a specific hospital (a real hospital or a digital twin hospital), the DTH extraction function 152 may extract a digital twin hospital corresponding to the hospital. In addition, the DTH extraction function 152 may extract a digital twin hospital of a general hospital when a clinical department is unknown or cannot be specified as one clinical department. In a case where the digital twin hospital meeting the condition cannot be extracted, the DTH extraction function 152 may output the fact.

The virtual medical consultation function 153 discloses the medical-related information of the user to the digital twin hospital, and acquires virtual medical consultation information generated by the virtual medical consultation for the user from the digital twin hospital. Here, the virtual medical consultation is performed in the digital twin hospital based on information regarding a real hospital corresponding to the digital twin hospital and medical-related information of the user.

Here, the information regarding the real hospital includes information regarding medical resources (medical facilities, devices, doctors, medical staffs, or the like) owned by the hospital. Furthermore, the information regarding the doctor of the medical resource may include information such as a medical practice, a treatment result for each treatment method, the number of experienced cases, and a specialty technique.

The virtual medical consultation information generated by the virtual medical consultation includes a diagnosis result by the virtual medical consultation and information (a treatment method, a treatment flow, a treatment result, or the like) regarding medical service that can be provided by a real hospital corresponding to the digital twin hospital that has received the virtual medical consultation. Note that, the virtual medical consultation information may include information regarding a medical treatment result that can be provided and a doctor belonging to a real hospital.

In a case where a plurality of digital twin hospitals have been extracted by the DTH extraction function 152, the virtual medical consultation function 153 may simultaneously or sequentially disclose user's medical-related information to the extracted plurality of digital twin hospitals and acquire the virtual medical consultation information from each digital twin hospital. It is possible to efficiently receive virtual medical consultation by simultaneously disclosing the medical-related information to the plurality of digital twin hospitals.

Note that, the virtual medical consultation function 153 may disclose the medical-related information only to the digital twin hospital permitted by the user among the plurality of digital twin hospitals extracted by the DTH extraction function 152. For example, information of the digital twin hospital (a corresponding real hospital location, a hospital visit time, or the like) extracted by the DTH extraction function 152 is displayed on the terminal 20, and the user selects the digital twin hospital that receives the virtual medical consultation from the presented digital twin hospital. The virtual medical consultation function 153 discloses the medical-related information only to the digital twin hospital selected by the user and causes the digital twin hospital to perform virtual medical consultation.

Note that, in a case where the virtual medical consultation result can be referred between the digital twin hospitals, there is a concern that some any changes may be made to the virtual medical consultation result. Therefore, the virtual medical consultation function 153 may not disclose the virtual medical consultation information acquired from a certain digital twin hospital to other digital twin hospitals. That is, the virtual medical consultation information acquired from the digital twin hospital may be provided only to the user who has received the virtual medical consultation.

The analysis function 154 generates analysis information based on a plurality of pieces of virtual medical consultation information acquired from a plurality of digital twin hospitals. The plurality of pieces of virtual medical care information acquired from the plurality of digital twin hospitals may be for virtual medical consultation received simultaneously or for virtual medical consultation performed at different timings.

The generated analysis information may be information for displaying treatment methods proposed by the plurality of digital twin hospitals in a comparable manner. In this case, the analysis function 154 generates analysis information by counting the number of answers for each treatment method proposed (answered) by the plurality of digital twin hospitals.

Further, the generated analysis information may be information for displaying the plurality of digital twin hospitals in a comparable manner. In this case, the analysis function 154 acquires a treatment result for each hospital from the virtual medical consultation information for a certain treatment method, and generates analysis information by comparing the treatment results.

The information output function 155 outputs various types of information. For example, the information output function 155 outputs the analysis information generated by the analysis function 154 to the terminal 20.

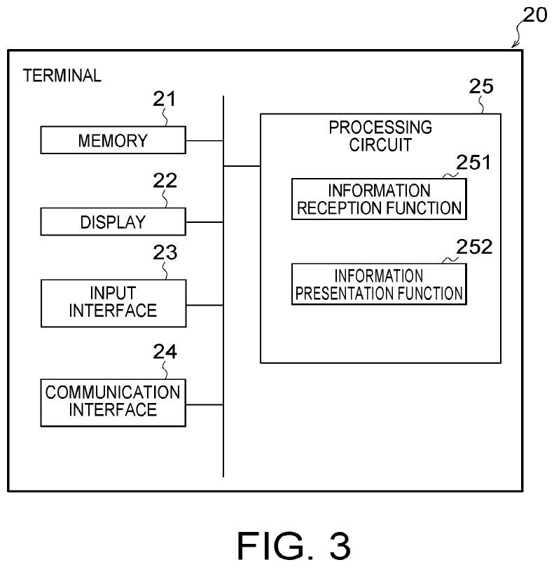

Next, details of the terminal 20 will be described with reference to FIG. 3. FIG. 3 is a block diagram illustrating an example of a configuration of the terminal 20 according to the present embodiment.

As illustrated in FIG. 3, the terminal 20 includes a memory 21, a display 22, an input interface 23, a communication interface 24, and a processing circuit 25.

The memory 21 is connected to the processing circuit 25, and stores various programs necessary for the processing circuit 25 to execute each function, various data processed by the programs, and the like. The memory 21 is realized by, for example, a random access memory (RAM), a semiconductor memory element such as a flash memory, a hard disk, an optical disk, or the like.

The display 22 displays various images and information. For example, the display 22 displays analysis information received from the server 10. Furthermore, the display 22 displays a graphical user interface (GUI) for receiving various operations from the user. In the present embodiment, the display 22 is configured by, for example, a liquid crystal display, a cathode ray tube (CRT) display, or the like. Note that, the display 22 may include a touch panel. In this case, the display 22 also serves as the input interface 23.

The input interface 23 receives various input operations from the user, converts the received input operations into electrical signals, and outputs the electrical signals to the processing circuit 25. The input interface 23 is realized by, for example, a mouse, a keyboard, a trackball, a manual switch, a foot switch, a button, a joystick, or the like.

The communication interface 24 implements various information communication protocols according to a form of the communication network N. The communication interface 24 realizes communication with other devices via the communication network N according to various protocols. In the present embodiment, the terminal 20 communicates with the server 10 via the communication interface 24.

The processing circuit 25 is an arithmetic circuit that performs various operations. The processing circuit 25 receives information and an instruction input by the user via the input interface 23, and displays information (analysis information and the like) received from the server 10 via the communication interface 24 on the display 22. In order to realize such a function, the processing circuit 25 has an information reception function 251 and an information presentation function 252 as illustrated in FIG. 3. The information reception function 251 is an example of an information reception unit, and information presentation function 252 is an example of an information presentation unit.

In the present embodiment, each processing function executed by the information reception function 251 and the information presentation function 252 is stored in the memory 21 in a form of a program executable by a computer. The processing circuit 25 is a processor, and implements a function corresponding to each program by reading and executing the program from the memory 21. In other words, the processing circuit 25 in a state of reading each program has each function illustrated in the processing circuit 25 of FIG. 3.

Note that, FIG. 3 illustrates a case where each processing function of the information reception function 251 and the information presentation function 252 is realized by the single processing circuit 25, but the embodiment is not limited thereto. For example, the processing circuit 25 may be configured by combining a plurality of independent processors, and each processor may implement each processing function by executing each program. Furthermore, each processing function of the processing circuit 25 may be implemented by being appropriately distributed or integrated into a single or a plurality of processing circuits.

The information reception function 251 receives information and an instruction input by the user via the input interface 23. The input information includes a DTH selection condition and the like. Furthermore, the user's instruction includes selection of a digital twin hospital from which the virtual medical consultation is received, and the like. Note that, the information reception function 251 may receive information necessary when the user creates an avatar in the virtual space.

The information presentation function 252 presents various types of information to the user by an output device such as the display 22. For example, the information presentation function 252 causes the display 22 to display the analysis information generated by the server 10.

### Operation Example of Medical Information Processing System

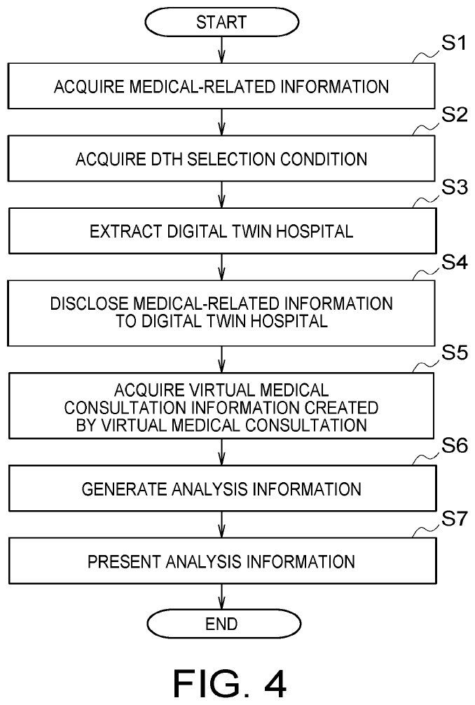

A medical information processing method using the above-described medical information processing system 1 will be described with reference to FIG. 4. FIG. 4 is a flowchart illustrating an example of a processing content executed by the medical information processing system 1.

In the following description, it is assumed that the user has created an avatar to which the medical-related information of the user is given in advance in the virtual space in which the digital twin hospital is set up. In this case, the medical-related information given to the avatar may be configured as a non-fungible token (NFT) using a technology such as blockchain in order to prove authenticity of data. In addition, the avatar (user) may also be anonymized. Also, the digital twin hospital in the virtual space may be anonymized.

First, the information acquisition function 151 of the server 10 acquires the medical-related information of the user (step S1). Here, the medical-related information of the user is acquired from the avatar.

Next, the information acquisition function 151 of the server 10 acquires the DTH selection condition set by the user from the terminal 20 (step S2).

Next, the DTH extraction function 152 of the server 10 extracts a plurality of digital twin hospitals based on the medical-related information acquired in step S1 and the DTH selection condition acquired in step S2 (step S3). Note that, the extracted digital twin hospital is not limited to one in which a corresponding real hospital is present in Japan, and may be one in overseas.

Next, the virtual medical consultation function 153 of the server 10 discloses the medical-related information acquired in step S1 to the digital twin hospital extracted in step S3 (step S4). For example, the virtual medical consultation function 153 discloses the medical-related information to all of the plurality of digital twin hospitals extracted in step S3.

Next, the virtual medical consultation function 153 of the server 10 acquires virtual medical examination information generated by virtual medical consultation from the digital twin hospital whose medical-related information is disclosed in step S4 (step S5). The digital twin hospital in which the medical-related information is disclosed in step S4 performs virtual medical consultation for the avatar based on the medical-related information and information regarding a real hospital corresponding to the digital twin hospital.

For example, the digital twin hospital outputs a medical treatment result, a virtual treatment flow, and a treatment result realized or predicted by the owned medical resources as virtual medical consultation information based on actually owned facilities, medical resources including doctors and medical technologies, and similar treatment experiences in the past. Since the owned facilities, the ability and experience values of the doctor, and the like are different for each hospital, the virtual treatment flow is created by comparing a realistic flow according to the actual situation of the hospital with the past treatment experience. In a case where there is no experience of a similar case, there may be a virtual medical consultation result with which an appropriate medical service cannot be provided. Furthermore, in a case where there are a plurality of doctors in the same hospital, since experience values, procedures, and the like are different for each doctor, one digital twin hospital may propose (answer) a virtual treatment flow for each doctor. Alternatively, only the virtual treatment flow with high accuracy by the doctor having the best treatment result may be answered.

Note that, in a case where the medical-related information is disclosed in a plurality of digital twin hospitals, the plurality of digital twin hospitals may simultaneously perform virtual medical consultation. The virtual medical consultation function 153 acquires virtual medical consultation information from each digital twin hospital.

Next, the analysis function 154 of the server 10 generates analysis information based on the virtual medical consultation information acquired in step S5 (step S6). The analysis information generated in this step is transmitted to the terminal 20 by the information output function 155.

The analysis information is, for example, information for displaying treatment methods proposed by a plurality of digital twin hospitals in a comparable manner. The analysis information may be information for displaying a plurality of digital twin hospitals in a comparable manner. Alternatively, the analysis information may be information for displaying doctors belonging to real hospitals corresponding to the plurality of digital twin hospitals in a comparable manner.

Note that, the analysis information may include a result of comparison of the treatment method answered from the digital twin hospital with standard clinical evidence or clinical practice guidelines. For example, the analysis information may include information indicating that a certain treatment method is a method recommended in the clinical practice guidelines.

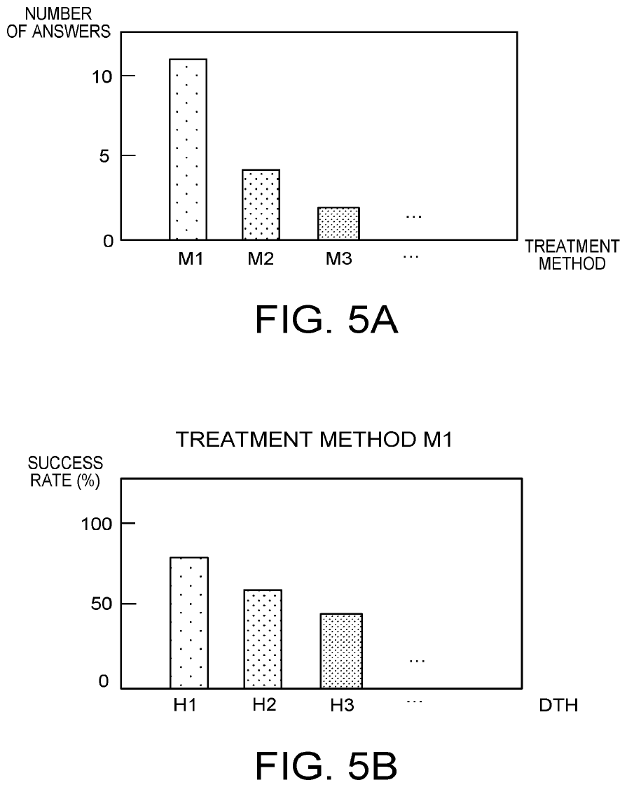

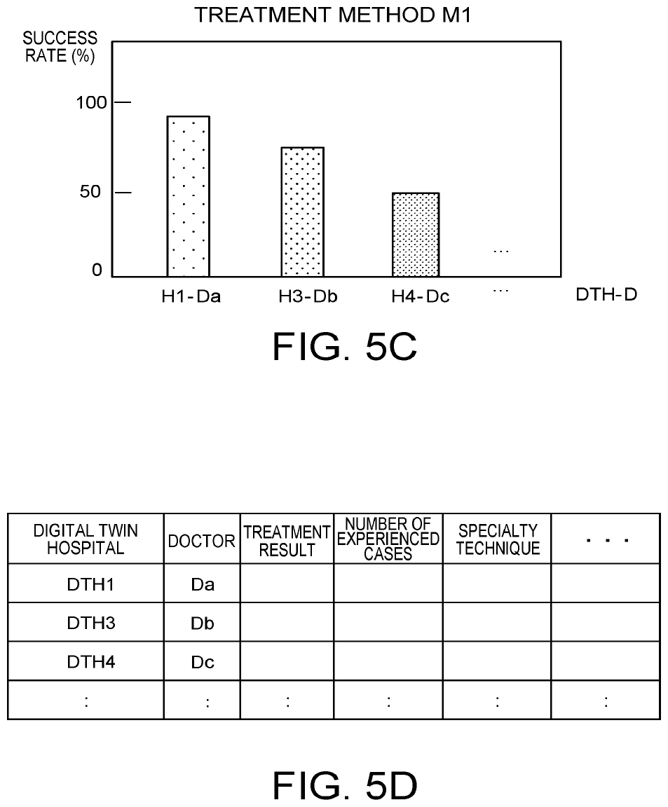

Next, the information presentation function 252 of the terminal 20 presents the analysis information received from the server 10 to the user (avatar) (step S7). Here, the information presentation function 252 causes the display 22 of the terminal 20 to display the analysis information. FIG. 5A to FIG. 5D illustrate examples of screens displayed on the terminal 20.

In FIG. 5A, the treatment methods answered from the plurality of digital twin hospitals are displayed as bar graphs in a descending order of the number of answers. In this example, the number of answers to a treatment method M1 is the largest, and is followed by treatment methods M2 and M3. As a result, the user can easily know what kind of treatment method is present, and can easily understand which treatment method is most common. In this manner, the treatment methods proposed by the plurality of digital twin hospitals are displayed on the terminal 20 in a comparable manner.

Note that, in the screen of FIG. 5A, it may be displayed that a certain treatment method (for example, the treatment method M2) is a method recommended in the clinical practice guidelines. As a result, the user can understand that the treatment method M2 is a standard treatment method although the number of answers is not the maximum.

In addition, the screen of FIG. 5A may display an evidence level of each treatment method. This allows the user to understand whether the treatment is a treatment method with low accuracy and with a small number of cases or a treatment method with high reliability and with sufficient evidence. By presenting the multifaceted analysis result in this manner, the user can obtain a more fair and multifaceted deep understanding of the presented treatment method.

In FIG. 5B, for a certain treatment method (here, the treatment method M1) among the proposed treatment methods, the digital twin hospital is displayed by a bar graph in a descending order of the treatment result (success rate). In this example, the digital twin hospital H1 has the highest success rate, and is followed by the digital twin hospitals H2 and H3. As a result, the user can easily know in which hospital the treatment method M1 can be received, and can easily understand which hospital has the best treatment result. In this manner, the plurality of digital twin hospitals are displayed on the terminal 20 in a comparable manner.

In FIG. 5C, for a certain treatment method (here, the treatment method M1) among the proposed treatment methods, doctors belonging to real hospitals corresponding to the plurality of digital twin hospitals are displayed by bar graphs in a descending order of treatment results (success rates). In FIG. 5C, for example, a doctor H1-Da represents a doctor Da belonging to a real hospital corresponding to a digital twin hospital H1. The same is true of the others. In this example, the doctor Da at the digital twin hospital H1 has the highest success rate, and is followed by a doctor Db at a digital twin hospital H3 and a doctor Dc at a digital twin hospital H4. As a result, the user can easily understand which doctor in which hospital can perform the treatment method M1, and can easily understand which doctor in which hospital has the best treatment result. As a result, the user can more accurately select a treatment method and select a hospital or a doctor.

Note that, the screen of FIG. 5B or FIG. 5C may be displayed by the user by selecting a treatment method on the screen of FIG. 5A. Furthermore, in order to prevent the user from being biased by a hospital name or the like, a hospital, a clinic department, and a doctor may be presented to the user in an anonymized state in FIG. 5B and FIG. 5C.

In addition, bar graphs are used in FIG. 5A to FIG. 5C, but the type of the graph may be another type such as a line graph or a circular graph.

In addition, a tabular format may be used without limitation to the graph display. FIG. 5D illustrates an example of a table corresponding to FIG. 5C. As in this example, detailed information (a treatment result, the number of experienced cases, a specialty technique, or the like) for each doctor may be displayed. Note that, in the display order of the digital twin hospitals and the doctors, the doctors may be displayed in a descending order of the treatment result from the top of the table, or may be displayed according to a predetermined order such as the Japanese syllabary order.

The above-described medical information processing method is merely an example, and various modifications can be made as follows.

For example, in a case where the user does not desire to set the DTH selection condition (in a case where the user desires to receive a virtual medical consultation without providing a special condition), step S2 may be omitted. In this case, the DTH extraction function 152 extracts the digital twin hospital only based on the medical-related information.

Furthermore, in step S3, the DTH extraction function 152 may extract the digital twin hospital designated by the user without considering the medical-related information. That is, the DTH extraction function 152 may extract the digital twin hospital only based on the DTH selection condition.

Furthermore, in step S4, the virtual medical consultation function 153 may disclose the medical-related information only to the digital twin hospital permitted by the user among the plurality of digital twin hospitals extracted in step S3. In this case, the server 10 transmits the information regarding the digital twin hospital (for example, the name of the digital twin hospital, the address of the corresponding real hospital, or the like) extracted in step S3 to the terminal 20. The terminal 20 displays the information regarding the digital twin hospital received from the server 10 on the display 22. The user selects the digital twin hospital for which the virtual medical consultation is desired. The virtual medical consultation function 153 discloses the medical-related information to the digital twin hospital selected by the user.

Furthermore, in a case where the desired hospital is not found, the user presented with the analysis information may change the DTH selection condition and receive the virtual medical consultation again. For example, in a case where a good result cannot be obtained at the digital twin hospital in Japan, the user may change the DTH selection condition so as to include a digital twin hospital overseas and receive the virtual medical consultation again. The user may correct the medical-related information and receive the virtual medical consultation again.

Furthermore, in order to establish the virtual medical consultation by the plurality of digital twin hospitals according to the embodiment as a business model, for example, virtual currency may be paid to the digital twin hospital (or an affiliated doctor) who has performed the virtual medical consultation. Furthermore, when the user decides to actually receive a medical consultation, personal information of the user may be disclosed to a real hospital in combination with virtual medical consultation information, and additional virtual currency may be further paid to the hospital to transfer to the real health insurance consultation.

As described above, in the present embodiment, the virtual medical consultation information generated by the virtual medical consultation for the user is acquired from the digital twin hospital in the virtual space, the analysis information is generated based on the plurality of pieces of virtual medical consultation information acquired from the plurality of digital twin hospitals, and the analysis information is presented to the user. As a result, the user can easily make a decision with a high sense of satisfaction such as which hospital and which doctor should be accessed and which treatment method should be adopted before actually receiving a medical consultation. That is, according to the present embodiment, it is possible to facilitate determination of selecting a hospital, a doctor, or a treatment method before actually receiving a medical consultation.

Note that the term “processor” used in the above description means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), and a field programmable gate array (FPGA)). The processor realizes the function by reading and executing the program stored in the memory 11. Note that, instead of storing the program in the memory 11, the program may be directly incorporated in a circuit of the processor. In this case, the processor realizes the function by reading and executing the program incorporated in the circuit. Note that, the processor is not limited to a case of being configured as a single processor circuit, and a plurality of independent circuits may be combined to be configured as one processor to realize the function.

Furthermore, the medical information processing method described with reference to FIG. 4 can be realized by executing a medical information processing program prepared in advance on a computer such as a personal computer or a workstation. The medical information processing program can be distributed via a network such as the Internet. The medical information processing program can also be executed by being recorded on a non-transitory computer-readable recording medium such as a hard disk, a flexible disk (FD), a CD-ROM, an MO, or a DVD and being read from the recording medium by the computer.

Although several embodiments have been described above, these embodiments have been presented only as examples, and are not intended to limit the scope of the invention. The novel devices and methods described herein can be implemented in a variety of other forms. In addition, various omissions, substitutions, and changes can be made to the forms of the apparatus and the method described in the present specification without departing from the gist of the invention. The appended claims and their equivalents are intended to include such forms and modifications as fall within the scope and spirit of the invention.

1. A medical information processing apparatus comprising processing circuitry configured to:
acquire virtual medical consultation information generated by a virtual medical consultation for a user from a digital twin hospital in a virtual space, wherein the virtual medical consultation is performed based on information regarding a real hospital corresponding to the digital twin hospital and medical-related information of the user;
generate analysis information based on a plurality of pieces of the virtual medical consultation information acquired from a plurality of the digital twin hospitals; and
present the analysis information to the user.

2. The medical information processing apparatus of claim 1, wherein the analysis information is information for displaying treatment methods proposed by the plurality of digital twin hospitals in a comparable manner.

3. The medical information processing apparatus of claim 2, wherein the analysis information is information for graphically displaying treatment methods proposed by the plurality of digital twin hospitals in a descending order of the number of answers.

4. The medical information processing apparatus of claim 1, wherein the analysis information is information for displaying the plurality of digital twin hospitals in a comparable manner.

5. The medical information processing apparatus of claim 4, wherein the analysis information is information for graphically displaying the plurality of digital twin hospitals in a descending order of treatment results for a certain treatment method among the treatment methods proposed by the plurality of digital twin hospitals.

6. The medical information processing apparatus of claim 1, wherein the analysis information is information for displaying doctors belonging to real hospitals corresponding to the plurality of digital twin hospitals in a comparable manner.

7. The medical information processing apparatus of claim 1, wherein the processing circuitry is further configured to extract at least one digital twin hospital that performs virtual medical consultation for the user among the plurality of digital twin hospitals set up in the virtual space based on a DTH selection condition and/or the medical-related information.

8. The medical information processing apparatus of claim 7, wherein the processing circuitry is further configured to disclose the medical-related information only to a digital twin hospital permitted by the user among the plurality of extracted digital twin hospitals.

9. The medical information processing apparatus of claim 7, wherein the processing circuitry is further configured to simultaneously disclose the medical-related information to the plurality of extracted digital twin hospitals.

10. The medical information processing apparatus of claim 1, wherein the medical-related information includes at least one of a main complaint, medical information, biological information, and behavior information of the user.

11. The medical information processing apparatus of claim 1, wherein the information regarding a real hospital includes information regarding medical resources owned by the hospital.

12. The medical information processing apparatus of claim 1, wherein the virtual medical consultation information includes a diagnosis result by the virtual medical consultation and information regarding medical service capable of being provided by a real hospital corresponding to the digital twin hospital.

13. The medical information processing apparatus of claim 1, wherein the processing circuitry is further configured not to disclose virtual medical consultation information acquired from a certain digital twin hospital to another digital twin hospital.

14. The medical information processing apparatus of claim 1, wherein the user and/or the digital twin hospital are anonymized.

15. The medical information processing apparatus of claim 1, further comprising a display configured to display the analysis information.

16. The medical information processing apparatus of claim 1, further comprising a storage to store the medical-related information.

17. A medical information processing method comprising:
acquiring virtual medical consultation information generated by virtual medical consultation for a user from a digital twin hospital in a virtual space, wherein the virtual medical consultation is performed based on information regarding a real hospital corresponding to the digital twin hospital and medical-related information of the user;
generating analysis information based on a plurality of pieces of the virtual medical consultation information acquired from a plurality of the digital twin hospitals; and
presenting the analysis information to the user.

18. A non-transitory computer-readable storage medium storing a program for causing a computer to execute a process, comprising:
acquiring virtual medical consultation information generated by virtual medical consultation for a user from a digital twin hospital in a virtual space, wherein the virtual medical consultation is performed based on information regarding a real hospital corresponding to the digital twin hospital and medical-related information of the user;
generating analysis information based on a plurality of pieces of the virtual medical consultation information acquired from a plurality of the digital twin hospitals; and
presenting the analysis information to the user.
